(19) United States
(12) Patent Application Publication
(10) Pub. No.: US 2025/0066663 A1
(43) Pub. Date: Feb. 27, 2025

(54) FLUORESCENT FIBERS FROM GREEN CHEMISTRY AND METHODS OF MAKING AND USING SAME

(71) Applicant: Saudi Arabian Oil Company, Dhahran (SA)

(72) Inventor: Wei Wang, Quincy, MA (US)

(21) Appl. No.: 18/452,934

(22) Filed: Aug. 21, 2023

(51) Int. Cl. C09K 11/65; G01N 33/28

(52) U.S. Cl. CPC C09K 11/65; G01N 33/2823

(57) ABSTRACT

The disclosure relates to fluorescent fibers and methods of making and using the fluorescent fibers. The fluorescent fibers can be made using natural sourced materials and green chemistry methods. The fluorescent fibers can be used as a tracer in mud logging applications by tagging drill cuttings with the fluorescent fibers at a drill bit during a drilling process, or as tracers in fracturing fluids for tracing the gas flow in a gas reservoir.

### FIELD

The disclosure relates to fluorescent fibers and methods of making and using the fluorescent fibers. The fluorescent fibers can be made using natural sourced materials and green chemistry methods. The fluorescent fibers can be used as a tracer in mud logging applications by tagging drill cuttings with the fluorescent fibers at a drill bit during a drilling process, or as tracers in fracturing fluids for tracing the gas flow in a gas reservoir.

### BACKGROUND

Drill cuttings obtained from mud logging can provide information on the formation lithology for geologic correlation, the mineral composition for marker beds, and input to corroborate data from other logging techniques. Mud tracers can be used to determine the cutting depths by mud cycle or circulation time. Mud return times of greater than half an hour can result in depth uncertainties of at least +/−20 feet using conventional mud logging service, which can propagate the errors in characterizing the formation according to depth.

### SUMMARY

The disclosure relates to fluorescent fibers and methods of making and using the fluorescent fibers. The fluorescent fibers can be made using natural sourced materials and green chemistry methods. The fluorescent fibers can be used as a tracer in mud logging applications by tagging drill cuttings with the fluorescent fibers at a drill bit during a drilling process, or as tracers in fracturing fluids for tracing the gas flow in a gas reservoir. Without wishing to be bound by theory, it is believed that cuttings depth assigned using the tracer tagged cuttings at the drill bit depth at the real time of the tag injection can significantly improve the depth correlation accuracy of cuttings to +/−2 feet in mud logging, relative to certain other methods.

The fluorescent fibers can be relatively easy and inexpensive to make. The fibers can be made from natural materials and using green chemistry methods. In general, the fluorescent fibers are relatively stable under ambient storage conditions and in reservoirs. For example, under such circumstances, the fluorescent fibers can remain stable for time periods on the span of days to weeks. Generally, the fluorescent fibers degrade under natural conditions on a time frame of days to months. The degradation process can be accelerated (e.g., to days to hours) using heat, sunlight and/or high or low pH conditions (e.g., at a pH above 10 or below 3).

The fibers are from natural materials (e.g., cotton, cellulose), and the fluorescence in the fluorescent fibers is imparted by carbon dots. Typically, the carbon dots are derived from a plant product (e.g., fruit juice, vegetable juice) or an animal product (e.g., egg whites, egg yolk, cow milk). Thus, the carbon dots can be a relatively inexpensive and environmentally benign fluorescent material. Compared to certain other fluorescent materials, such as fluorescent semiconductor quantum dots, carbon dots according to the disclosure can exhibit improved fluorescent properties, such as relatively broad excitation spectra, narrow and tunable emission spectra, and high photostability against photo bleaching and blinking.

The fluorescent fibers can be used as mud logging tracers. For example, the fluorescent fibers can be embedded into or disposed on (e.g., permanently disposed on) the drill cuttings when the mud fluids contact the formation at the drill bit (see discussion below). Compared to certain other logging methods, fluorescent fibers according to the disclosure are more environmentally friendly and naturally degradable after use, and can be orthogonally detected by multiple analytical methods.

Without wishing to be bound by theory, it is believed that conventional mud tracers only tag mud fluid and travel with the mud flow back to surface for detection in mud samples whereas the fluorescent fibers can tag cuttings at the drill bit, then travel together with the cuttings back to the surface for detection in the cutting samples. Thus, the fluorescent fibers can give more accurate information on the depth at which the cuttings are formed relative to certain other mud tracers.

In a first aspect the disclosure provides a composition, including a plurality of fibers, each fiber including a plurality of fluorescent carbon dots. The fluorescent carbon dots are derived from a component of a plant, an egg, or a milk.

In some embodiments, the fibers include cotton fibers and/or wood fibers.

In some embodiments, the fluorescent carbon dots are derived from a component of fruit juice, vegetable juice, egg whites and/or egg yolks, cow and/or goat milk.

In some embodiments, the fibers include a lumen and at least a portion of the fluorescent carbon dots are disposed in the lumen of the fibers. In some embodiments, the fibers include mesopores and at least a portion of the fluorescent carbon dots are disposed in the mesopores of the fibers. In some embodiments, the fibers include an external surface and at least a portion of the fluorescent carbon dots are disposed on the external surface.

In some embodiments, the fibers have a length of from 0.1 mm to 3.5 cm.

In some embodiments, the fibers are mechanically cut or ground or freeze-ground into micro-fibers with a sub-millimeter length.

In some embodiments, the fibers have a diameter of from 8 μm to 30 μm.

In some embodiments, the fluorescent carbon dots emit at a wavelength of from 300 nm to 700 nm.

In some embodiments, the fluorescent carbon dots have a diameter of from 5 nm to 30 nm.

In a second aspect, the disclosure provides a method, including flowing a solution including fibers of the disclosure into an underground formation during drilling of the underground formation with a drill bit.

In certain embodiments, the fibers attach to rock chips formed during the drilling at the drill bit.

In some embodiments, the method further includes: collecting the rock chips; measuring a fluorescence of the rock chips; and correlating a depth at which the rock chips were formed based on the measured fluorescence.

In a third aspect, the disclosure provides a method, including flowing a fluid including the fibers disclosed herein into a gas reservoir.

In certain embodiments, the method further includes: producing a gas from the gas reservoir; measuring a fluorescence of the produced gas; and correlating a location at which the fibers were triggered to be released into gas, based on the measured fluorescence.

In certain embodiments, the fluid is a component in a flooding process.

In certain embodiments, the fluid includes a proppant and the fibers are attached to the proppant.

In a fourth aspect, the disclosure provides a method, including: contacting a plurality of fibers with a member selected from the group consisting of fruit juice, vegetable juice, egg whites and egg yolks, milks, the member including fluorescent carbon dot precursors, thereby forming a plurality of fibers including the fluorescent carbon dot precursors, each fiber including a plurality of fluorescent carbon dot precursors; and heating the plurality of fibers including the fluorescent carbon dot precursors, thereby converting the fluorescent carbon dot precursors into fluorescent carbon dots and forming a plurality of fibers including fluorescent carbon dots, each fiber including a plurality of fluorescent carbon dots.

In some embodiments, the fibers include cotton fibers and/or wood fibers.

In some embodiments, the heating is performed at a temperature of 150 to 190° C.

In some embodiments, the fibers include a lumen, prior to the heating, at least a portion of the fluorescent carbon dot precursors are disposed in the lumen of the fibers, and after the heating, at least a portion of the fluorescent carbon dots are disposed in the lumen of the fibers. In some embodiments, the fibers include mesopores, prior to the heating, at least a portion of the fluorescent carbon dot precursors are disposed in the mesopores of the fibers, and after the heating, at least a portion of the fluorescent carbon dots are disposed in the mesopores of the fibers. In some embodiments, the fibers include an outer surface, prior to the heating, at least a portion of the fluorescent carbon dot precursors are disposed on the outer surface of the fibers, and after the heating, at least a portion of the fluorescent carbon dots are disposed on the outer surface of the fibers.

In some embodiments, the fluorescent carbon dots emit at a wavelength of from 300 nm to 700 nm.

### BRIEF DESCRIPTION OF THE FIGURES

FIG. 1A depicts a schematic of a partial cutaway longitudinal view of a fluorescent fiber.

FIG. 1B depicts a schematic of a cross-sectional view of a fluorescent fiber.

FIG. 1C depicts a schematic of a cross-sectional view of a fluorescent fiber.

FIG. 2 depicts a schematic of a flow chart.

FIG. 3 depicts a schematic of a system.

FIGS. 4A-4D depict scanning electron microscope (SEM) images of fluorescent fibers.

FIG. 5A depict fluorescent spectra of fluorescent fibers.

FIGS. 5B-5C depict two-dimensional fluorescent spectral profiles of fluorescent fibers.

FIGS. 6A-6B depict fluorescence images of fluorescent fibers.

### DETAILED DESCRIPTION

### Fluorescent Fibers

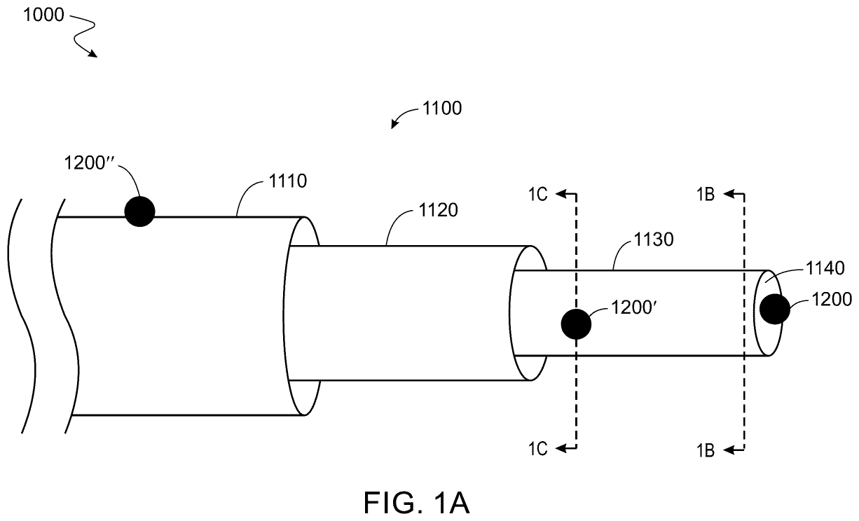

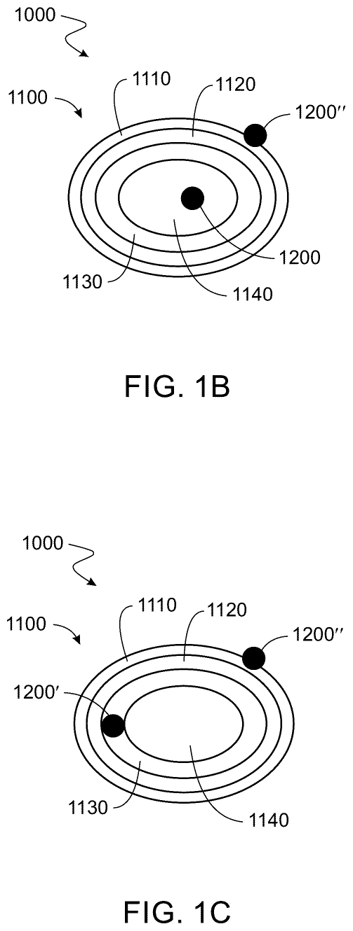

FIG. 1A depicts a partial cutaway longitudinal view of a schematic of a fluorescent fiber 1000. FIGS. 1B and 1C depict schematics of cross-sectional views of the fluorescent fiber 1000 along the lines 1B-1B and 1C-1C, respectively. The fluorescent fibers include a cotton fiber 1100 and carbon dots 1200.

The cotton fiber 1100 is hollow and includes a multilayer structure with a cuticle 1110, a primary wall 1120 within the cuticle 1110, and a secondary wall 1130 within the primary wall 1120. The secondary wall 1130 defines a lumen 1140. The cotton fiber 1100 has a fibrillar structure. The secondary wall 1130, containing about 92-95% cellulose, includes concentric layers with alternating shaped twists. The layers include densely packed elementary fibrils, organized into micro fibrils and macro fibrils and are held together by hydrogen bonds. The lumen 1140 forms the center of the cotton fiber 1100.

In FIG. 1A, one of the carbon dots 1200 is within the lumen 1140, another carbon dot 1200′ is within the mesopores of the cotton fiber 1100 (e.g., the mesopores of the secondary wall 1130), and another carbon dot 1200″ is on an outer surface of the cotton fiber 1100. The carbon dots 1200, 1200′ and 1200″ are fluorescent and can demonstrate good fluorescent properties, such as relatively broad excitation spectra, relatively narrow and tunable emission spectra, and relatively high photostability against photobleaching and blinking.

While untreated cotton fibers are non-emissive or weakly emissive, the fluorescent fibers can be emissive under appropriate excitation light. For example, under ultraviolet (UV, 365 nm) excitation light, cotton fibers treated with orange juice, egg white and cow milk (see discussion below), exhibit emission maximum at ˜450 nm, ˜440 nm and ˜460 nm, respectively.

In certain embodiments, the fluorescent fibers 1000 have a length in the range of millimeters to centimeters, for example, at least 1 (e.g., at least 2, at least 5, at least 10, at least 15, at least 20, at least 25, at least 30) mm and/or at most 35 (e.g., at most 30, at most 25, at most 20, at most 15, at most 10, at most 5, at most 2) mm. In certain embodiments, the fluorescent fibers 1000 have a diameter of at least 8 (e.g., at least 10, at least 12, at least 14, at least 16, at least 18) μm and/or at most 20 (e.g., at most 18, at most 16, at most 14, at most 12, at most 10) μm.

In certain embodiments, the fluorescent fibers 1000 can be mechanically cut or ground to micro-fibers with a sub-millimeter length, for example, at most 1 (e.g., at most 0.5, at most 0.25, at most 0.1) mm.

In addition or in alternative to cotton fibers 1100, the fluorescent fibers 1000 can include other cellulose containing materials that include a hollow structure, such as wood fibers.

The carbon dots 1200 are derived from a plant product (e.g., fruit juice, vegetable juice) or an animal product (e.g., egg whites). The carbon dots 1200 typically have size below 30 nm and an sp²-conjugated structure surrounded by oxygen-containing groups such as hydroxyl and carboxyl groups, and exhibit relatively good physicochemical features such as relatively low toxicity, relative chemical inertness, biocompatibility, and relatively high photostability.

In general, the plant product can include any fruit juice. Examples of plant products include juice from lime, kiwi, grape, avocado, carrot, mango, apple, pear, peach, and/or orange. Without wishing to be bound by theory, it is believed that fluorescent fibers prepared with different starting materials (e.g., different fruit juices, vegetable juices, egg whites and/or yolks, milks) can result in fluorescent fibers with different fluorescent properties, corresponding to different excitation and emission wavelengths, which can be identified by fluorescence methods.

In some embodiments, the carbon dots 1200 have an excitation maximum of at least 260 (e.g., at least 280, at least 300, at least 320, at least 340, at least 350, at least 360, at least 380, at least 400, at least 420, at least 440, at least 450, at least 460, at least 480, at least 500, at least 520, at least 540) nm and/or at most 550 (e.g., at most 540, at most 520, at most 500, at most 480, at most 460, at most 450, at most 440, at most 420, at most 400, at most 380, at most 360, at most 350, at most 340, at most 320, at most 300, at most 280) nm. In some embodiments, the carbon dots 1200 have an emission maximum of at least 300 (e.g., at least 320, at least 340, at least 350, at least 360, at least 380, at least 400, at least 420, at least 440, at least 450, at least 460, at least 480, at least 500, at least 520, at least 540, at least 550, at least 560, at least 580, at least 600, at least 620, at least 640) nm and/or at most 650 (e.g., at most 640, at most 620, at most 600, at most 580, at most 560, at most 550, at most 540, at most 520, at most 500, at most 480, at most 460, at most 450, at most 440, at most 420, at most 400, at most 380, at most 360, at most 350, at most 340, at most 320) nm. In some embodiments, the carbon dots 1200 have a diameter of at least 5 (e.g., at least 10, at least 15, at most 20, at least 25) nm and/or at most 30 (e.g., at most 25, at most 20, at most 15, at most 10) nm.

### Method of Making Fluorescent Fibers

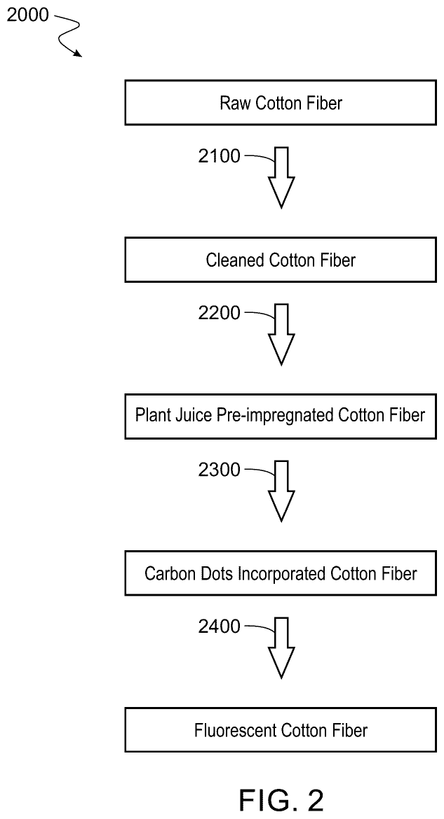

FIG. 2 depicts a flow chart for a method 2000 of making fluorescent fibers.

In step 2100, raw cotton fiber is ultrasonicated in dilute base (e.g., NaOH, KOH) and washed with water to provide cleaned cotton fiber. In certain embodiments, the concentration of base is 0.01 (e.g., at least 0.02, at least 0.05, at least 0.1, at least 0.15) M and/or at most 0.2 (e.g., at most 0.15, at most 0.1, at most 0.05, at most 0.02) M.

In step 2200, pulp-free plant juice (e.g., fruit juice, vegetable juice) and cleaned cotton fibers are ultrasonicated to allow the carbon dot precursors (e.g., citric acid and/or sugar as a carbon source) to infiltrate into the cotton fibers (e.g., into the lumen and/or mesopores of the cotton fibers) to yield plant juice pre-impregnated cotton fibers. In addition or in alternative to pulp free-plant juice, the step 2200 can include egg whites and/or yolks, cow or goat milk, which contain proteins and amino acids as carbon dot precursors.

In step 2300, the plant juice pre-impregnated cotton fibers are autoclaved for carbonization of the carbon precursors to form carbon dots in and on the cotton fibers, and yield cotton fibers with incorporated carbon dots. In some embodiments, the autoclaving is performed at a temperature of at least 150 (e.g., at least 155, at least 160, at least 165, at least 170, at least 175, at least 180, at least 185) ° C. and/or at most 190 (e.g., at most 185, at most 180, at most 175, at most 170, at most 165, at most 160, at most 155) ° C. In some embodiments, the autoclaving is performed at 160° C. for 2 hours. Without wishing to be bound by theory, the step 2300 leads to the hydrothermal formation of carbon dots by the carbonization of the carbon dot precursors (e.g., citric acids, sugars, proteins, amino acids).

In step 2400, the cotton fibers with incorporated carbon dots are purified and dried to yield fluorescent cotton fibers. The purification can include rinsing with water and an alcohol (ethanol, methanol). The drying can include drying at a temperature of at least 50 (e.g., at least 60, at least 70, at least 80, at least 90, at least 100, at least 110) ° C. and/or at most 120 (e.g., at most 110, at most 100, at most 90, at most 80, at most 70, at most 60) ° C. In some embodiments, the drying is performed at a temperature of 60° C. In some embodiments, the drying is performed in air.

The methods described above can be used for other appropriate cellulose containing materials other than cotton fibers, such as wood fibers.

### Fluorescent Fibers as Mud Logging Tracers

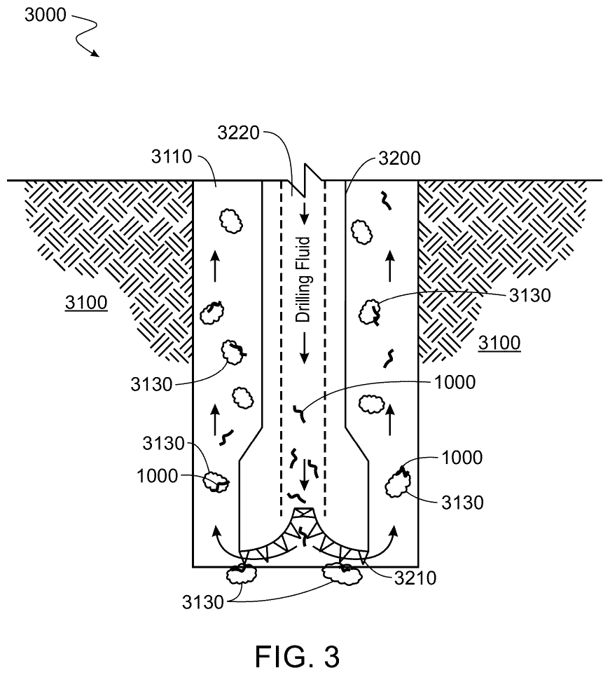

FIG. 3 depicts a schematic of a system 3000. The system includes an underground formation 3100 and a borehole 3110 extending into the underground formation 3100. A drill 3200 is used to drill the borehole 3110 into the underground formation 3100. The drill 3200 includes a drill bit 3210 to drill into the underground formation 3100 and a drill pipe 3220 through which a drilling fluid can be sent during use of the drill bit 3120. As the drill bit cuts into the underground formation 3100, rock chips 3130 are generated. The drilling fluid is used to aid in the drilling of the borehole 3110 and carry cuttings out of the borehole 3110. Fluorescent fibers 1000 are included in the drilling fluid. Without wishing to be bound by theory, it is believed that the fluorescent fibers 1000 are mechanically cut by the drill bit 3120 and embedded into to the rock chips 3130 (e.g., into the pores of the rock chips 3130) by mechanical force and physical adsorption, thereby tagging the rock chips 3130 based on the depth. The depth is evaluated base the inner diameter of the drill pipe 3220, and injection rate and volume of the drill fluid at certain time intervals. The rock chips 3130 are then carried to the surface by the drilling fluid and collected. Fibers created using different precursors (e.g., different fruit and vegetable juices, egg whites, egg yolks, milks) may exhibit fluorescence at different excitations/emissions, and thus can be identified as barcoded tracers, when pulsed injected at different times, correlating to different depths. The depth at which rock chips 3130 were formed can be determined from the fluorescent properties of the fluorescent fibers 1000.

Collected rock chips 3130 can be screened on site using a fluorescent measurement to identify rock chips 3130 tagged with fluorescent fibers 1000. The presence of the fluorescent fibers 1000 can be detected by fluorescent imaging with a fluorescence microscope, a high-resolution fluorescence camera, or a portable fluorescence spectrometer for relatively rapid on-site screening. Rock chips 3130 tagged with fluorescent fibers 1000 can be brought to a laboratory for a more detailed analysis with an in vivo imaging system (IVIS), high-resolution fluorescence spectroscopy, or scanning electron microscope (SEM).

The rock chips 3130 selected for more detailed analysis can be washed (e.g., with water and/or ethanol or methanol) to remove mud and unattached fluorescent fibers 1000 and analyzed. Fluorescent fibers synthesized by different methods can be identified using fluorescent imaging with a fluorescence microscope, a high-resolution fluorescence camera, or an in vivo imaging system (IVIS), or by spectroscopy with a portable fluorescence spectrometer.

In certain embodiments, the concentration of fluorescent fibers in the drilling fluid is 0.01 (e.g., at least 0.02, at least 0.05, at least 0.1, at least 0.2, at least 0.5) wt. % and/or at most 1.0 (e.g., at most 0.5, at most 0.2, at most 0.1, at most 0.05, at most 0.02) wt. %. The drilling fluid can be a water-based or oil-based drilling mud fluid.

### Fluorescent Fibers as Gas Tracers

The fluorescent fibers can be used as a gas tracer in a gas reservoir (e.g., a shale gas reservoir). Without wishing to be bound by theory, it is believed that the fluorescent fibers have a sufficiently low density such that the fluorescent fibers can flow with a produced gas. The fluorescent fibers can be mechanically cut or ground into small pieces with size in length of millimeters or sub-millimeter and introduced into the gas reservoir with a fluid injected into the gas reservoir, such as a fluid used in a flooding or hydraulic fracturing process. As gas is produced from the reservoir (e.g., from a gas production well), the fluorescent fibers can travel with the gas and then be collected from the produced gas by filtration and then analyzed by a fluorescence method. Without wishing to be bound by theory, it is believed that based on the fluorescent measurements a location at which the fibers were triggered to be released into the gas can be determined.

In certain embodiments, the fluorescent fibers can be injected into the gas reservoir with a fluid, such as a hydraulic fracturing fluids, during a flooding process. The fluids used are typically a liquid but can include gases, such as carbon dioxide. In certain embodiments, the fluorescent tags can be attached onto proppants by degradable polymers or resins during a hydraulic fracturing process, and gas or oil can trigger the polymer to release the fibers into the gas. The proppants can include sand or ceramic materials with grain size from 0.1 mm to 2.5 mm.

### EXAMPLES

### Example 1

0.5 g of cotton fiber was pretreated with 0.1 M sodium hydroxide under ultrasonication, then rinsed with deionized water. Kiwi juice was collected from squeezed fresh kiwi, and filtered using a 0.45 micron syringe filter to remove pulp. The pulp-free juice was diluted with deionized water at a 1:1 volume ratio. The pretreated cotton fiber and 20 mL diluted pulp-free kiwi juice were mixed under ultrasonication for 30 min to allow the juice solution to infiltrate into the cotton fibers. The mixture was transferred into a Teflon-lined stainless steel autoclave and the hydrothermal synthesis reaction was allowed to proceed at 160° C. for 2 hours. The obtained cotton fibers were washed with deionized water after the hydrothermal reaction, rinsed with ethanol or methanol, and dried at 60° C. in air.

The procedure described above was repeated with pulp-free orange juice. The procedure described above was also repeated with ground micro-fibers of cotton. When viewed under a fluorescence microscope with an excitation of 365 nm UV lamp, bright fluorescence was observed for the cotton fibers synthesized from the pulp-free fruit juices.

### Example 2

0.5 g of cotton fiber was pretreated with 0.1 M sodium hydroxide solution under ultrasonication, then rinsed with deionized water. Egg yolk and whites were separated from a fresh egg, and the egg whites were collected and mixed with deionized water at 1:1 volume ratio. The pretreated cotton fiber and 20 mL diluted egg whites solution were mixed under ultrasonication for 30 min to allow the egg protein to infiltrate into the cotton fibers. The mixture was transferred into a Teflon-lined stainless steel autoclave and the hydrothermal synthesis reaction was allowed to proceed at 160° C. for 2 hours. The obtained cotton fibers were washed with deionized water after the hydrothermal reaction, then rinsed with alcohol, and finally the fibers were dried at 60° C. in air.

The procedure described above was repeated with egg yolk. The procedure described above was also repeated with ground micro-fibers of cotton. When viewed under a fluorescence microscope with an excitation of 365 nm UV lamp, bright fluorescence was observed for the cotton fibers synthesized from the eggs.

### Example 3

0.5 g of cotton fiber was pretreated with 0.1 M sodium hydroxide solution under ultrasonication, then rinsed with deionized water. Cow whole milk was mixed with deionized water at 1:1 volume ratio. The pretreated cotton fiber and 20 mL diluted milk solution were mixed under ultrasonication for 30 min to allow the milk protein to infiltrate into the cotton fibers. The mixture was transferred into a Teflon-lined stainless steel autoclave and the hydrothermal synthesis reaction was allowed to proceed at 160° C. for 2 hours. The obtained cotton fibers were washed with deionized water after the hydrothermal reaction, then rinsed with alcohol, and finally the fibers were dried at 60° C. in air.

The procedure described above was repeated with goat milk. The procedure described above was also repeated with ground micro-fibers of cotton. When viewed under a fluorescence microscope with an excitation of 365 nm UV lamp, bright fluorescence was be observed for the cotton fibers synthesized from the milks.

### Example 4

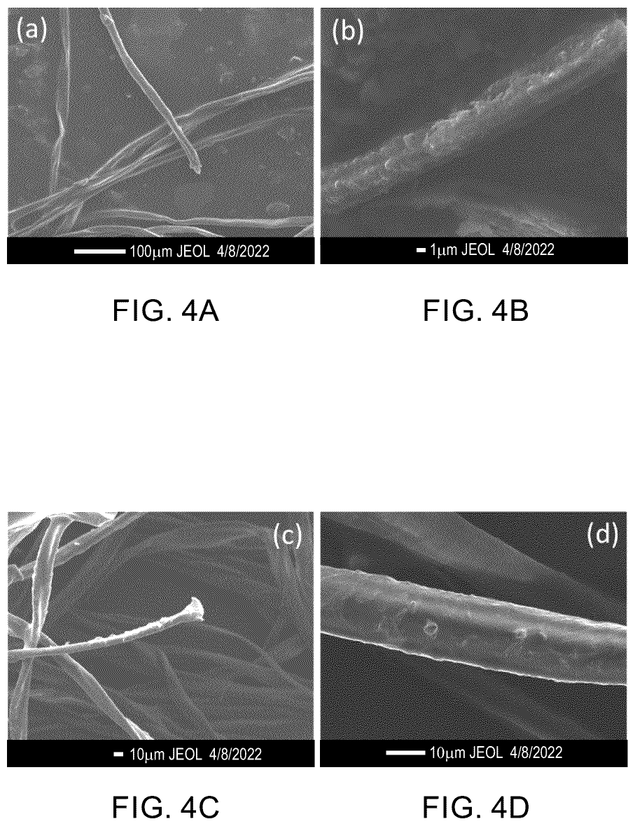

The fluorescent fibers prepared in Examples 1 and 2 were measured using scanning electron microscopy (SEM). Images were recorded using a JEOL JSM-7100F field emission SEM instrument 15 kV voltage. FIGS. 4A and 4B show SEM images of the fluorescent fibers prepared in Example 1 and FIGS. 4C and 4D show SEM images of the fluorescent fibers prepared in Example 2.

The SEM images showed that the carbon dots were self-assembled on the surface of the cotton fibers.

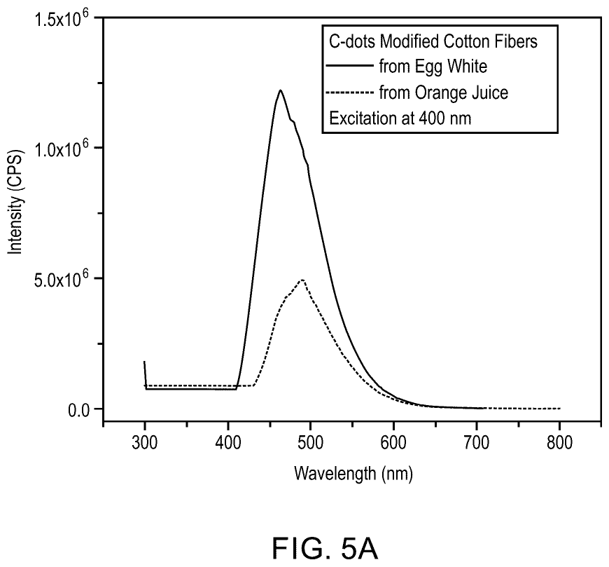

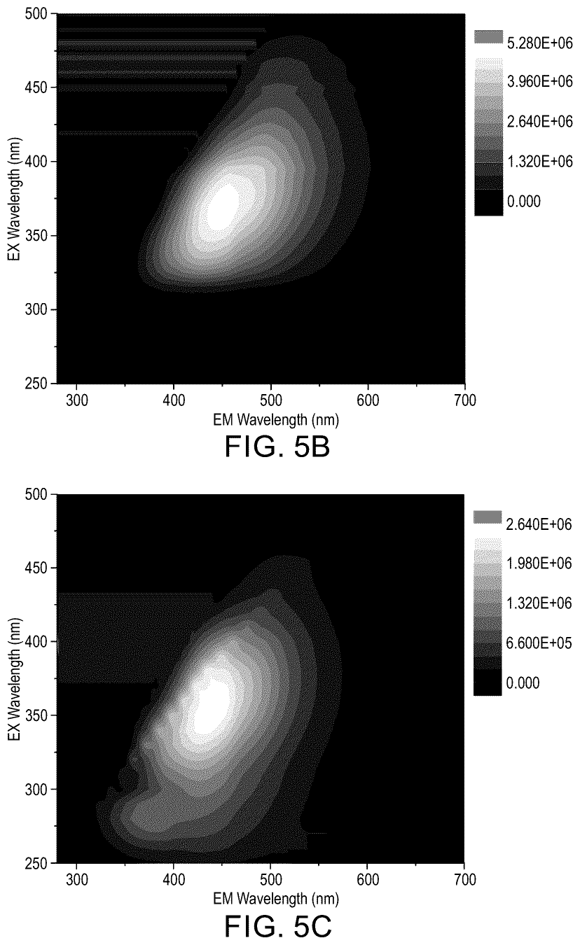

One- and two-dimensional (2D) fluorescence spectra of the fluorescent cotton fibers prepared in Examples 1, 2 and 3 were measured using a Horiba Nanolog-FL3-22iHR spectrometer with 450W xenon arc lamp and R928P PMT detector at 400 nm excitation. FIG. 5A shows the fluorescence spectra of the fluorescent cotton fibers prepared with orange juice in Examples 1 and with egg white in Example 2. FIGS. 5B and 5C show the 2D fluorescence spectral profiles of the fluorescent cotton fibers prepared with orange juice in Examples 1 and with egg white in Example 2, respectively.

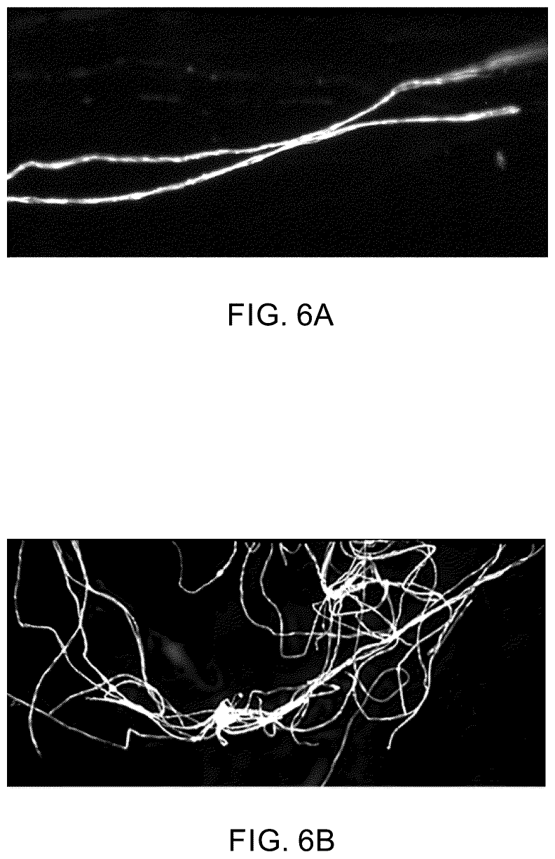

Fluorescent images of the fibers made using orange juice and egg white under 365 nm excitation are presented in FIGS. 6A and 6B, respectively.

What is claimed:

1. A composition, comprising:
a plurality of fibers, each fiber comprising a plurality of fluorescent carbon dots,
wherein the fluorescent carbon dots are derived from a component of a plant, an egg, or a milk.

2. The composition of claim 1, wherein the fibers comprise a member selected from the group consisting of cotton fibers and wood fibers.

3. The composition of claim 1, wherein the fluorescent carbon dots are derived from a component of a member selected from the group consisting of fruit juice, vegetable juice, egg whites, egg yolks, cow milk and goat milk.

4. The composition of claim 1, wherein at least one of the following holds:
the fibers comprise a lumen and at least a portion of the fluorescent carbon dots are disposed in the lumen of the fibers;
the fibers comprise mesopores and at least a portion of the fluorescent carbon dots are disposed in the mesopores of the fibers; or
the fibers comprise an external surface and at least a portion of the fluorescent carbon dots are disposed on the external surface.

5. The composition of claim 1, wherein the fibers have a length of from 0.1 mm to 3.5 cm.

6. The composition of claim 1, wherein the fibers have a diameter of from 8 μm to 30 μm.

7. The composition of claim 1, wherein the fluorescent carbon dots emit at a wavelength of from 300 nm to 700 nm.

8. The composition of claim 1, wherein the fluorescent carbon dots have a diameter of from 5 nm to 30 nm.

9. A method, comprising:
flowing a solution comprising the fibers of claim 1 into an underground formation during drilling of the underground formation with a drill bit.

10. The method of claim 9, wherein the fibers attach to rock chips formed during the drilling at the drill bit.

11. The method of claim 10, further comprising:
collecting the rock chips;
measuring a fluorescence of the rock chips; and
correlating a depth at which the rock chips were formed based on the measured fluorescence.

12. A method, comprising:
flowing a fluid comprising the fibers of claim 1 into a gas reservoir.

13. The method of claim 12, further comprising:
producing a gas from the gas reservoir;
measuring a fluorescence of the produced gas; and
correlating a location at which the fibers were triggered to be released into gas, based on the measured fluorescence.

14. The method of claim 12, wherein the fluid is a component in a flooding process.

15. The method of claim 12, the fluid comprises a proppant and the fibers are attached to the proppant.

16. A method, comprising:
contacting a plurality of fibers with a member selected from the group consisting of fruit juice, vegetable juice, egg whites and egg yolks, milk, the member comprising fluorescent carbon dot precursors, thereby forming a plurality of fibers comprising the fluorescent carbon dot precursors, each fiber comprising a plurality of fluorescent carbon dot precursors; and
heating the plurality of fibers comprising the fluorescent carbon dot precursors, thereby converting the fluorescent carbon dot precursors into fluorescent carbon dots and forming a plurality of fibers comprising fluorescent carbon dots, each fiber comprising a plurality of fluorescent carbon dots.

17. The method of claim 16, wherein the fibers comprise a member selected from the group consisting of cotton fibers and wood fibers.

18. The method of claim 16, wherein the heating is performed at a temperature of 150 to 190° C.

19. The method of claim 16, wherein at least one of the following holds:
the fibers comprise a lumen, prior to the heating, at least a portion of the fluorescent carbon dot precursors are disposed in the lumen of the fibers, and after the heating, at least a portion of the fluorescent carbon dots are disposed in the lumen of the fibers;
the fibers comprise mesopores, prior to the heating, at least a portion of the fluorescent carbon dot precursors are disposed in the mesopores of the fibers, and after the heating, at least a portion of the fluorescent carbon dots are disposed in the mesopores of the fibers; or
the fibers comprise an outer surface, prior to the heating, at least a portion of the fluorescent carbon dot precursors are disposed on the outer surface of the fibers, and after the heating, at least a portion of the fluorescent carbon dots are disposed on the outer surface of the fibers.

20. The method of claim 16, wherein the fluorescent carbon dots emit at a wavelength of from 300 nm to 700 nm.
